(19) United States
(12) Patent Application Publication
(10) Pub. No.: US 2004/0267163 A1
(43) Pub. Date: Dec. 30, 2004

(54) Medical guide wires

(76) Inventors: John C. Opie, Scottsdale, AZ (US); Stephen J. Joyce, Phoenix, AZ (US); Thomas Izdebski, Phoenix, AZ (US)

Correspondence Address: SQUIRE, SANDERS & DEMPSEY L.L.P., Two Renaissance Square, Suite 2700, 40 North Central Avenue, Phoenix, AZ, 85004-4498, US

(21) Appl. No.: 10/861,888

(22) Filed: Jun. 3, 2004

Related U.S. Application Data
(60) Provisional application No. 60/475,666, filed on Jun. 3, 2003.

(51) Int. Cl. A61M 25/09

(52) U.S. Cl. 600/585

(57) ABSTRACT

Disclosed is a medical guide wire having a center portion and a floppy, or flexible and relatively soft, tail attached to each end of the center portion. Each floppy tail can be of any configuration, length or diameter. Also disclosed are optically-enhanced medical guide wires and medical guide wires with a smooth outer surface. The optically-enhanced medical guide wires preferably glow under ultraviolet light but can be optically enhanced in any manner that makes them highly visible during medical procedures. The smooth outer surface on a medical guide wire reduces friction and can be moved more easily through the vascular system, and are easier to clean than wires with a matte finish.

### RELATED APPLICATIONS

This application claims priority to U.S. Provisional Application Ser. No. 60/475,666, filed Jun. 3, 2003 and entitled “Improved Medical Guide Wires.”

### FIELD OF THE INVENTION

This invention relates to the field of medical devices and, more particularly, to improved medical guide wires having two flexible (or floppy) ends, an optically-enhanced coating and/or a smooth surface to reduce friction.

### BACKGROUND OF THE INVENTION

One aspect of the invention relates to medical guide wires and how they interact with their surroundings, including deep blue medical drapes, very low light conditions in typical endo-vascular procedure labs, and the problem of blood clots drying on the surface of the guide wires. Guide wires are utilized for advancing endovscular-intraluminal devices such as diagnostic catheters, balloon angioplasty systems, stent delivery devices, atherectomy catheters and the like within the body. In a typical percutaneous procedure utilizing a guide wire, a guiding needle is percutaneously introduced into a patient's peripheral artery, e.g., femoral or brachial artery, by means of a conventional Seldinger technique. Once an intraluminal location is confirmed an opening guide wire is passed through the needle into the vessel. This guide wire is typically 018″ in diameter. Once that is passed up the artery the needle is removed and a sheath is placed over the 018″ wire. Once the sheath is placed, typically the 0.018″ mandril wire is removed and placed under a wet lap sponge. A regular 0.035″, 4.5 mm J×140 cm long Benson guide wire is typically selected and passed up the sheath and is positioned say in the lower aorta. Once the Benson guide wire is confirmed as being in place in the blood vessel then a diagnostic or therapeutic catheter is passed over the Benson guide wire. The Benson guide wire is then removed and is wiped clean and stored under a wet lap sponge. If the lesion under treatment cannot be passed with the Benson then frequently a Glide wire straight, curved or “J,” may be used. Once the lesion is crossed, then a PTA or stent can be deployed using a catheter guided along the guide wire.

If a guide wire exchange (whereby one guide wire is exchanged for another) is necessary for a multiplex of reasons, then a long catheter is passed over the Glide wire. The Glide wire is then removed from the blood vessel, the catheter is maintained in the blood vessel, and perhaps the Benson or an Amplatz wire for example, is inserted or re-inserted. Before doing so, the re-inserted wire is wiped with a wet sponge but it is difficult to see if all of the blood clots on the wire's surface in the low light conditions. Sometimes, because all wires tend to appear as the same generally dark color, some temporary confusion occurs as to where each type of wire is temporarily stored. Furthermore, the user's vision is sometimes if wearing glasses and/or a protective plastic face mask.

The use of intraluminal catheters for various endovascular procedures is well known. In order to get the endovascular catheter to the desired location within the vasculature it is necessary first to manipulate a guide wire into the proper place in a blood vessel. Then utilizing selected preformed wire shapes it is usually possible to slide a catheter over the guide wire and to the selected position. At that point some form of diagnostic or therapeutic procedure is performed.

Some general interest references that teach methods of manufacturing guide wires are U.S. Pat. Nos. 5,484,419, 4,813,938 and 5,045,065, the respective disclosures of which that teach guide wire manufacturing and materials are incorporated herein by reference.

Existing guide wires usually have a single highly flexible and relative soft and (referred to herein as a floppy end). These guide wires have several drawbacks. The first is in facilitating loading the guide wire using automatic wire feeders. In this situation, a guide wire with a rigid end, when it is loaded into a spiral package, impacts the outer circumference of the inner spiral chamber and after one or two loops are made the wire binds and cannot be further advanced into the spiral chamber. With a floppy tail (and perhaps a specialized spiral) the floppy tail should allow the wire to be machine fed completely into the spiral chamber without binding. The second drawback is operator protection. Many guide wires are long, up to 9-10 feet in length, and some of the guide wires are quite stiff (such as Amplatz and Lindiquist wires). If such a wire were to get loose and flick out of its temporary containment during temporary non-use, the rear end of the wire could accidentally injure an unprotected eye or face. Such a misfortune could result in either severe corneal abrasion or permanent blindness if eyeball penetration were to occur.

Current guide wires have matte finishes due to the textures of the coatings applied to the wires. Unfortunately blood clots and other debris adhere to the textured surface. By providing a smooth coating, there is no textured surface to adhere to and less chance of blood sticking to the guide wire.

### SUMMARY OF THE INVENTION

The invention relates to a guide wire having one or more of the following characteristics: (1) a floppy, or flexible, tail at each end, (2) being optically-enhanced so as to be relatively easy to se in operating room conditions, and (3) having a smooth surface to lessen friction when being moved through the vaculative.

Optically-enhanced guide wires would lessen the problem of locating wires during operations since they would be highly visible, and preferably distinct from each other (since different types of guide wires could be given different colors) and the presence of surface blood clots on such guide wires would be easily seen and the wire could be properly wiped clean before re-insertion into a blood vessel. While many colors could be used, such colors as bright green, pink, white and yellow, or some combination of these or other bright colors, are considered useful high-intensity colors and would fit the requirements of possessing high visibility and making it easy to see blood clots.

Also, of significance, is the rear end of the guide wire. There are several reasons for making all guide wires with two floppy tails instead of one. The second floppy tail helps to protect the user and support persons from being struck in the face or the eye with a sharp end. By adding the second floppy tail the risk of such injuries will be minimized. Another reason for adding a second floppy tail is to provide on occasion, when a wire might become bent near the tip, it would now be possible to reverse the wire and use the other end. Further, the guide wire could contain two different floppy tails and a different end of the same wire could be used for different procedures.

### BRIEF DESCRIPTION OF THE DRAWINGS

FIG. 1 is a cross sectional view of a guide wire with a floppy tail having a coil core;

FIG. 2 is a cross sectional view of a guide wire with a floppy tail having an epoxy core;

FIG. 3 is a cross sectional view of a guide wire with a floppy tail formed from a plastic coating; and

FIG. 4 is a section of guide wire formed with a optically-enhanced coating represented by cross-hatch shading.

### DETAILED DESCRIPTION OF PREFFERED EMBODIMENTS

### Embodiment 1

Optically-Enhanced Guide Wire

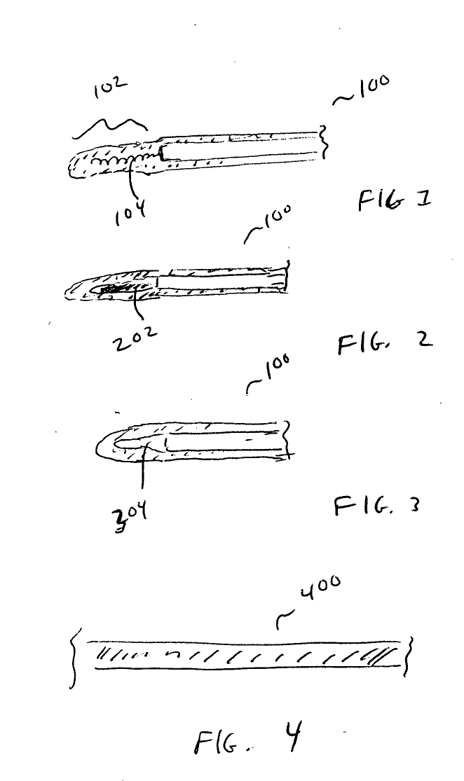

FIG. 1 shows guide wires with shading representing an optically-enhanced coating.

The external plastic coatings already applied to most guide wires could be colored to make the optically-enhanced guide wire. Any applied coating may be optically enhanced. Additional transparent or semi-transparent coatings, such as hydrophilic coatings, could be coated over the optically-enhanced coating.

Optically-enhanced coatings could also be applied to a guide wire in any conventional manner such as vacuum deposition, spray coating, UV curing processes or the like. The optically-enhanced coating could be hydrophilic or the guide wire, after being coated with an optically-enhanced coating, can be coated with a transparent hydrophilic coating if desired. In one embodiment, the optically-enhanced coating is applied as a smooth coating.

### Embodiment 2

Double Floppy Tail Guide Wire

Referring now to FIGS. 1 and 2, a longitudinal cross section view of the floppy tail is provided. The floppy tail consists of a central core of a stainless steel helical coil. The floppy tail is covered with a suitable plastic coating that also covers the guide wire itself. Such coatings include floppy tails, with or without helical stainless steel coils, made of platinum or a tantalum filled epoxy, which may or may not be a hydrophobically coated, have multiple polymer coatings, and intermediate sections with a flexible core, which like the floppy tip is radio-opaque. Such wires may have different grades of polymer coating including but not limited to such substances as polyurethane 55D and 90A or polytetrafluoroethylene, or silicone, or may have single polymeric coating with varying properties along its length with or without lubricious or hydrophilic coatings. Referring to FIG. 3, whatever external plastic coating used to cover the guide wire, such as a Benson wire or Amplatz wire or Glide wire for example, in this instance the coating is simply extended past the rear end of the wire and made into a floppy tail extension.

Numerous characteristics and advantages of the invention covered in this document have been set forth in the foregoing description. It will be understood, however, that this disclosure only illustrative. Changes may be made in details, particularly with respect to shape, size, and length of the floppy tail, without exceeding the scope of the invention.

### Embodiment 3

Smooth Surface Guide Wire

A “smooth coating” means that the exterior surface of the guide wire is generally smooth and does not have the textures (raised and lowered portions), such as a matt finish found on typical guide wire surfaces. Unfortunately blood clots and other debris adhere to the textured surface. By providing a smooth coating, there is no textured surface to adhere to and less chance of blood sticking to the guide wire. A smooth coating and its method of manufacture are known to those skilled in the art. The coating can be a plastic coating, a paint coating or any other type of coating. Having now described preferred embodiments of the invention, modifications and variations to the present invention may be made by those skilled in the art. The invention is thus not limited to the preferred embodiments, but is instead set forth in the following claims and legal equivalents thereof.

What is claimed is:

1. A guide wire for use in a medical procedure, the guide wire having a central portion having a first end and a second end and further comprising a first floppy tail attached to the first end, the first end and first floppy tail for insertion into a patient, and a second floppy tail attached to the second end.

2. The guide wire of claim 1 wherein the first floppy tail has a proximal end coupled to the first end and a distal end.

3. The guide wire of claim 1 wherein the flexible central section comprises a steel coil.

4. The guide wire of claim 1 wherein the central portion has a plastic coating and the first floppy tail is made by extending the plastic coating of the central portion beyond the first end.

5. The guide wire of claim 1 wherein the floppy tail has a steel care and a plastic coating.

6. The guide wire of claim 1 wherein the central portion has a plastic coating and the second floppy tail is made by extending the plastic coating of the central portion beyond the second end.

7. The guide wire of claim 1 wherein the first floppy tail is straight and the second floppy tail is C-shaped.

8. The guide wire of claim 1 wherein the first floppy tail is straight and the second floppy tail is straight.

9. The guide wire of claim 1 wherein the first floppy tail is straight and the second floppy tail is J-shaped.

10. The guide wire of claim 1 wherein the first floppy tail is C-shaped and the second floppy tail is J-shaped.

11. The guide wire of claim 1 that has a diameter of 0.018″.

12. The guide wire of claim 1 that has a diameter of 0.034″.

13. The guide wire of claim 1 wherein each floppy tail has a diameter and the central portion has a diameter, the diameter of each floppy tail is equal to the diameter of the central portion.

14. The guide wire of claim 1 wherein the first floppy tail is equal in length to the second floppy tail.

15. The guide wire of claim 1 wherein the first floppy tail is longer than the second floppy tail.

16. The guide wire of claim 1 wherein the first floppy tail is of the same stiffness as the second floppy tail.

17. An optically-enhanced medical guide wire comprising a core and an optically-enhanced material, the guide wire capable of being easily seen by operating room personnel during ht periods where there is little visible light.

18. The guide wire of claim 17 that comprises an optically-enhanced coating.

19. The guide wire of claim 17 wherein the guide wire glows when exposed to ultraviolet light.

20. The guide wire of claim 18 wherein the enhanced coating is a paint applied to the guide wire.

21. The guide wire of claim 18 wherein the guide wire comprises an optically-enhanced plastic film applied to the guide wire.

22. The guide wire of claim 20 wherein the plastic film is vacuum formed to the guide wire.

23. A guide wire having a smooth outer surface.

24. A guide wire for use in a medical procedure, the guide wire comprising:
an outer surface, the outer surface being smooth;
an optically-enhanced coating on the outer surface; and
a central portion having a first end and a second end, and a first floppy tail attached to the first end and a second floppy tail attached to the second end.

25. The guide wire of claim 24 wherein the first floppy tail is 3 mm in length.

26. The guide wire of claim 24 wherein the first floppy tail is 5 mm in length.

27. The guide wire of claim 24 wherein the second floppy tail is 3 mm in length.

28. The guide wire of claim 24 wherein the second floppy tail is 5 mm in length.
